(19) United States
(12) Patent Application Publication
(10) Pub. No.: US 2007/0172509 A1
(43) Pub. Date: Jul. 26, 2007

(54) Drug Delivery System for Retarding Release of Water Soluble Drugs

(75) Inventors: Thai Minh Nguyen, Santa Clara, CA (US); Theodore L. Parker, Danville, CA (US); John F. Shanley, Redwood City, CA (US)

Correspondence Address: CINDY A. LYNCH;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/626,785

(22) Filed: Jan. 24, 2007

Related U.S. Application Data
(60) Provisional application No. 60/761,645, filed on Jan. 24, 2006.

(51) Int. Cl. A61K 31/727; A61K 31/65; A61K 31/60; A61K 31/519; A61K 38/28

(52) U.S. Cl. 424/426; 514/165; 514/56; 514/262.1; 514/152; 514/3

(57) ABSTRACT

An implantable drug delivery system uses a hydrophobic compound as an outer layer or barrier for retarding release of water soluble drugs from the implantable system. The system includes an inner portion of a water soluble drug in a drug matrix material which stabilizes the drug. An outer portion of the drug delivery system separates the inner portion from a surrounding environment. The outer portion retards the release of the water soluble drug from the inner portion. The outer portion includes a hydrophobic non-polymer compound and a binder. The hydrophobic compound can be another drug which can be delivered at an entirely different release kinetic from the water soluble drug and for treatment of the same or a different condition. When the drug delivery system is implanted in a body the outer portion retards the release of the water soluble drug by controlling fluid passing from the body into the inner portion and by controlling passage of the water soluble drug from the inner portion into the body.

### RELATED APPLICATIONS

This application claims priority to U.S. Provisional Patent Application Ser. No. 60/761,645, filed Jan. 24, 2006, the entire contents of which are incorporated herein by reference.

### FIELD OF THE INVENTION

The invention relates to a therapeutic agent delivery system for the controlled release of water soluble therapeutic agents.

### DESCRIPTION OF THE RELATED ART

Implantable medical devices are often used for delivery of a beneficial agent, such as a drug, to an organ or tissue in the body at a controlled delivery rate over an extended period of time. These devices may deliver agents to a wide variety of bodily systems to provide a wide variety of treatments.

One of the many implantable medical devices which have been used for local delivery of beneficial agents is the coronary stent. In order to provide local delivery of drugs from stents, the surface of the stent is coated with a combination of drug and polymer. Surface coatings, however, can provide little actual control over the release kinetics of beneficial agents. These coatings are necessarily very thin, typically 5 to 8 microns thick. The surface area of the stent, by comparison is very large, so that the entire volume of the beneficial agent has a very short diffusion path to discharge into the surrounding tissue.

Increasing the thickness of the surface coating has the beneficial effects of improving drug release kinetics including the ability to control drug release and to allow increased drug loading. However, the increased coating thickness results in increased overall thickness of the stent wall which is undesirable. In addition to sub-optimal release profiles, there are further problems with surface coated stents. The permanent polymer carriers frequently used in the device coatings can retain a large amount of the beneficial agent in the coating indefinitely. Since these beneficial agents are frequently highly cytotoxic, sub-acute and chronic problems such as chronic inflammation, late thrombosis, and late or incomplete healing of the vessel wall may occur. Additionally, the carrier polymers themselves are often highly inflammatory to the tissue of the vessel wall.

Another significant problem with drug/polymer coatings is that expansion of the stent may stress the overlying polymeric coating causing the coating to plastically deform, to rupture, or to separate from the underlying stent surface. Separation of a coating may result in uneven drug delivery and even embolization of coating fragments causing vascular obstruction.

In addition, it is not currently possible to deliver some drugs with a surface coating for a variety of reasons. In some cases, the drugs are sensitive to water, other compounds, or conditions in the body which degrade the drugs. For example, some drugs lose substantially all their activity when exposed to water for a period of time. When the desired treatment time is substantially longer than the half life of the drug in water the drug cannot be delivered by know coatings. Other drugs, such as protein or peptide based therapeutic agents, lose activity when exposed to enzymes, pH changes, or other environmental conditions.

Drugs that are highly-soluble in water are particularly problematic when delivered from coated implantable devices. These water soluble drugs tend to be released from surface coatings at an undesirably high rate and do not remain localized for a therapeutically useful amount of time.

Accordingly, it would be desirable to provide an implantable drug delivery device for delivery of water soluble drugs to a patient while protecting the agent from fluids in the body which would cause the drug to quickly wash out of the coating.

### SUMMARY OF THE INVENTION

In accordance with one aspect of the invention, an implantable drug delivery system for retarding release of water soluble drugs comprises an inner portion of the drug delivery system comprising a water soluble drug and a drug matrix material which stabilizes the drug, and an outer portion of the drug delivery system which retards the release of the water soluble drug from the inner portion, the outer portion comprising a hydrophobic non-polymer compound and less than 50% of a binder, wherein when the drug delivery system is implanted in a body the outer portion retards the release of the water soluble drug by controlling fluid passing from the body into the inner portion and by controlling passage of the water soluble drug from the inner portion into the body.

In accordance with a second aspect of the invention, a drug delivery stent comprises an expandable stent structure having a plurality of reservoirs, a drug delivery system provided within the reservoirs of the stent structure, the drug delivery system having an inner portion and an outer portion wherein the inner portion of the drug delivery system comprises a water soluble drug and a drug matrix material which stabilizes the drug and wherein the outer portion of the drug delivery system retards the release of the water soluble drug from the inner portion, the outer portion comprising a hydrophobic non-polymer compound and of a binder at a ratio of less than 50% by weight of the binder, wherein when the stent is implanted in a body the outer portion retards the release of the water soluble drug by controlling fluid passing from the body into the inner portion and by controlling passage of the water soluble drug from the inner portion into the body.

In accordance with another aspect of the invention a drug delivery stent comprises an expandable stent structure having a plurality of reservoirs, a drug delivery system provided within the reservoirs of the stent structure, the drug delivery system having an inner portion and an outer portion wherein the inner portion of the drug delivery system comprises a water soluble drug and a drug matrix material which stabilizes the drug and wherein the outer portion of the drug delivery system retards the release of the water soluble drug from the inner portion, the outer portion comprising a hydrophobic non-polymer compound and of a binder at a ratio of less than 50% by weight of the binder, wherein when the stent is implanted in a body the outer portion retards the release of the water soluble drug by controlling fluid passing from the body into the inner portion and by controlling passage of the water soluble drug from the inner portion into the body.

In accordance with an additional aspect of the invention, a drug delivery stent comprises an expandable stent structure, a drug delivery system secured to the stent structure, the drug delivery system having an inner portion and an outer portion wherein the inner portion of the drug delivery system comprises a water soluble drug and a drug matrix material which stabilizes the drug and wherein the outer portion of the drug delivery system retards the release of the water soluble drug from the inner portion, the outer portion comprising a hydrophobic non-polymer compound, wherein when the stent is implanted in a body the outer portion retards the release of the water soluble drug by controlling fluid passing from the body into the inner portion and by controlling passage of the water soluble drug from the inner portion into the body.

### BRIEF DESCRIPTION OF THE DRAWINGS

The invention will now be described in greater detail with reference to the preferred embodiments illustrated in the accompanying drawings, in which like elements bear like reference numerals, and wherein:

FIG. 1 is a perspective view of one example of a stent according to the present invention.

FIG. 2 is a side view of a portion of the stent of FIG. 1.

FIG. 3 is a side cross sectional view of an example of an opening in a medical device showing a drug delivery system within a reservoir in the medical device.

FIGS. 4a and 4b are graphs of the release curves for insulin and Pimecrolimus from the dual drug stent described in Example 1.

FIGS. 5a and 5b are graphs of the release curves for insulin and Pimecrolimus from the dual drug stent described in Example 2.

FIGS. 6a and 6b are graphs of the release curves for insulin and Pimecrolimus from the dual drug stent described in Example 3.

FIGS. 7a and 7b are graphs of the release curves for insulin and Pimecrolimus from the dual drug stent described in Example 4.

FIGS. 8a and 8b are graphs of the release curves for insulin and Pimecrolimus from the dual drug stent described in Example 5.

FIGS. 9a and 9b are graphs of the release curves for insulin and Pimecrolimus from the dual drug stent described in Example 6.

### DETAILED DESCRIPTION

An implantable drug delivery system uses a hydrophobic compound as an outer layer or barrier for retarding release of water soluble drugs from the implantable system. The system includes an inner portion of a water soluble drug in a drug matrix material which stabilizes the drug. An outer portion of the drug delivery system separates the inner portion from a surrounding environment. The outer portion retards the release of the water soluble drug from the inner portion. The outer portion includes a hydrophobic non-polymer compound and a binder. The hydrophobic compound can be another drug which can be delivered at an entirely different release kinetic from the water soluble drug and for treatment of the same or a different condition. When the drug delivery system is implanted in a body the outer portion retards the release of the water soluble drug by controlling fluid passing from the body into the inner portion and by controlling passage of the water soluble drug from the inner portion into the body.

In one example described in detail herein the water soluble drug and the hydrophobic compound will be contained in reservoirs in a stent body prior to release. In the reservoir example, the water soluble drug and the hydrophobic material can both be combined with matrices, such as bioresorbable polymers to hold the compounds within the reservoirs in the stent.

The following terms, as used herein, shall have the following meanings:

The terms “drug” and “therapeutic agent” are used interchangeably to refer to any therapeutically active substance that is delivered to a living being to produce a desired, usually beneficial, effect.

The term “matrix” or “biocompatible matrix” or “binder” are used interchangeably to refer to a medium or material that, upon implantation in a subject, does not elicit a detrimental response sufficient to result in the rejection of the matrix. The matrix may contain or surround a therapeutic agent, and/or modulate the release of the therapeutic agent into the body. A matrix is also a medium that may simply provide support, structural integrity or structural barriers. The matrix may be polymeric, non-polymeric, hydrophobic, hydrophilic, lipophilic, amphiphilic, and the like. The matrix may be bioresorbable or non-bioresorbable.

The term “bioresorbable” refers to a matrix, as defined herein, that can be broken down by either chemical or physical process, upon interaction with a physiological environment. The matrix can erode or dissolve. A bioresorbable matrix serves a temporary function in the body, such as drug delivery, and is then degraded or broken into components that are metabolizable or excretable, over a period of time from minutes to years, usually less than one year, while maintaining any requisite structural integrity in that same time period.

The terms “openings” and “reservoirs” include both through openings and recesses of any shape.

The term “Pharmaceutically acceptable” refers to the characteristic of being non-toxic to a host or patient and suitable for maintaining the stability of a therapeutic agent and allowing the delivery of the therapeutic agent to target cells or tissue.

The term “polymer” refers to molecules formed from the chemical union of two or more repeating units, called monomers. Accordingly, included within the term “polymer” may be, for example, dimers, trimers, oligomers and copolymers prepared from two or more different monomers. The polymer may be synthetic, naturally occurring or semisynthetic. The term “polymer” refers to molecules which have a Mw greater than about 3000 and preferably greater than about 10,000 and a Mw that is less than about 10 million, preferably less than about a million and more preferably less than about 200,000. Examples of polymers include but are not limited to, poly-α-hydroxy acid esters such as, polylactic acid (PLLA or DLPLA), polyglycolic acid, polylactic-co-glycolic acid (PLGA), polylactic acid-co-caprolactone; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclodextrin sulfobutyl ethers; polypeptides and proteins, such as polylysine, polyglutamic acid, albumin; polyanhydrides; polyhydroxy alkonoates such as polyhydroxy valerate, polyhydroxy butyrate, and the like.

The term “non-polymer” refers to molecules which are not formed from the chemical union of two or more repeating units, called monomers or to molecules which have a Mw less than about 3000.

The term “primarily” with respect to directional delivery, refers to an amount greater than 50% of the total amount of therapeutic agent provided to a blood vessel.

The term “restenosis” refers to the renarrowing of an artery following an angioplasty procedure which may include stenosis following stent implantation. Restenosis is a wound healing process that reduces the vessel lumen diameter by extracellular matrix deposition, neointimal hyperplasia, and vascular smooth muscle cell proliferation, and which may ultimately result in renarrowing or even reocclusion of the lumen.

The term “anti-restenotic” refers to a drug which interferes with any one or more of the processes of restenosis to reduce the renarrowing of the lumen.

The term “hydrophobic” refers to a compound which has a calculated Log P or Log D value of at least one, where P is the octanol to water partition coefficient and D is the octanol to water coefficient at a specified pH value.

The term “water soluble” refers to a compound whose solubility in water is greater than about 1 mg per milliliter.

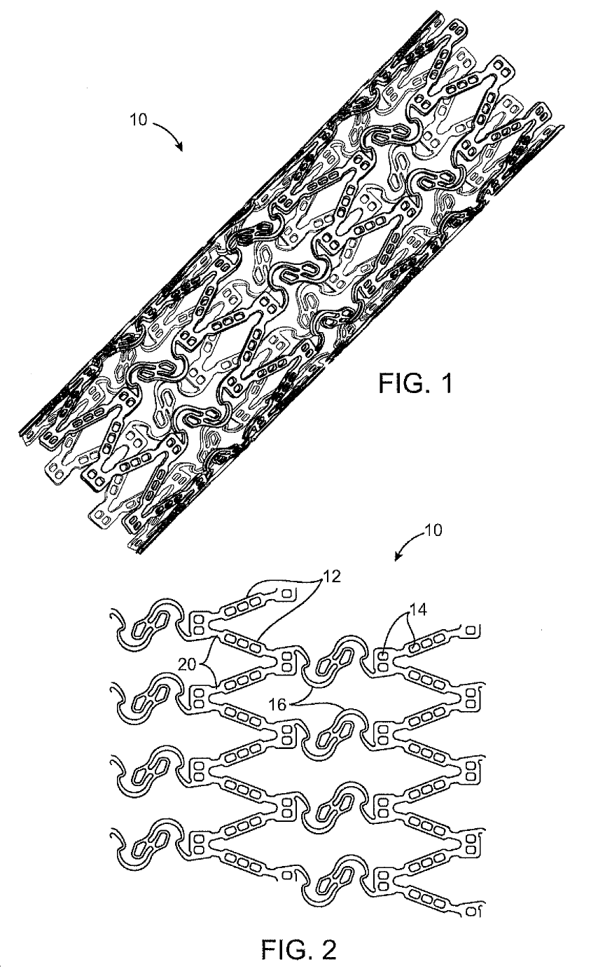

FIG. 1 illustrates one example of an implantable medical device in the form of a stent 10. FIG. 2 is an enlarged flattened view of a portion of the stent of FIG. 1 illustrating one example of a stent structure including struts 12 interconnected by ductile hinges 20. Bridging elements 16 provide axial flexibility to the stent structure. The struts 12 and various other substantially non-deforming structures within the stent include openings 14 containing a therapeutic agent. The openings 14 are preferably non-deforming openings. One example of a stent structure having non-deforming openings is shown in U.S. Pat. No. 6,562,065 which is incorporated herein by reference in its entirety.

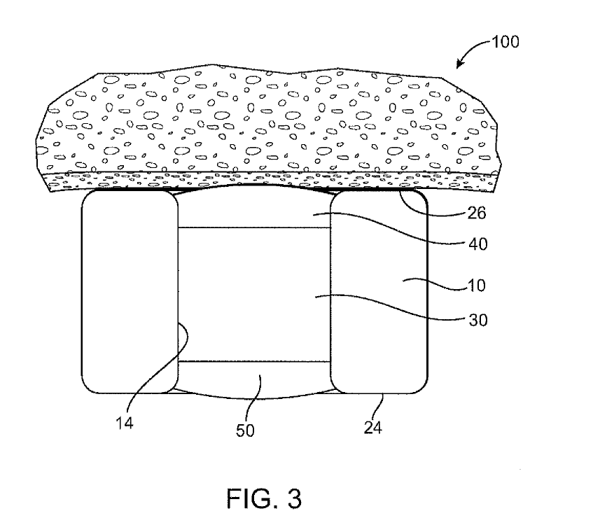

FIG. 3 illustrates one example of a reservoir system for a stent or other implantable medical device. FIG. 3 shows a cross section through one strut of a stent 10 with a luminal surface 24, a mural surface 26, and an opening 14. Within the opening 14, one example of an inlay is shown. The inlay includes an inner portion 30 which includes the water soluble drug in a drug matrix material. The inlay inner portion 30 is covered on one or both of the luminal and mural ends of the opening by an outer portion 40 which retards the release of the water soluble drug by controlling fluid passing from the body into the inner portion 30 and by controlling passage of the water soluble drug from the inner portion into the body. In the example shown in FIG. 3, the outer portion 40 is at the mural side of the stent and the luminal side of the stent is provided with a base portion 50. The outer portion 40 includes a hydrophobic non-polymer compound, such as a hydrophobic drug and a minor amount of a binder.

Although the inner portion 30, outer portion 40, and base portion 50 have been illustrated as discrete layers, it is understood that these portions, depending on the method of fabrication may be commingled at their margins resulting in a continuously changing inlay composition The configuration in which a drug and other compounds can be precisely arranged within the reservoir allows the release rate and administration period for release of the drug to be selected and programmed to a particular application. An example of some of the methods which can be used to precisely arranged the drug within the matrix in the openings include a stepwise deposition process which is further described in U.S. patent Publication 2004-0073294, which is incorporated herein by reference.

Conventional bioresorbable polymers, such as PLGA, used as rate controlling portions in implantable drug delivery systems have difficulty in minimizing the burst and sustaining the release of water soluble drugs, or the dose of the water soluble drug must be greatly reduced to achieve a low burst and/or sustained delivery. By increasing the hydrophobicity of the rate controlling cap, barrier, or other rate controlling portion, the rate of water ingress and water soluble drug elution from the drug matrix can be retarded.

Drugs that are sensitive to decomposition or inactivation during storage in a drug delivery device require that the medium immediately surrounding them, the so-called “drug matrix material”, actively stabilizes the drug, or at least does not act to promote degrade or inactivation. This is accomplished either by the inherent physical and chemical properties of the matrix material, or by inclusion of stabilizing additives in the matrix composition. It is often the case that within the overall composition of the sustained delivery device, the material that is most suitable as a matrix for the drug is not also the most suitable for obtaining sustained release of the drug, particularly when the drug is water soluble. The drug delivery system of the present invention allows the drug matrix material to be specifically selected for its stabilizing properties but not its drug delivery properties. While an outer portion is formulated with a hydrophobic non-polymeric compound to retard the release of the water soluble drug in the mural direction (where the composition is a “cap deposit”) and/or in the luminal direction (where the composition is a “base deposit”) release. Consequently, the water soluble drug can be disposed in a matrix specifically designed for the function of protecting the drug during storage, and the controlled release of the drug can be accomplished with a different matrix material designed to retard the drug release.

For example, insulin is a protein drug that is highly water soluble and is sensitive not only to chemical degradation, but also to bio-inactivation by a change in conformation. A saccharide matrix for the water soluble drug can be used that stabilizes insulin, but because it is itself water soluble, it cannot retard the release of insulin. Other compounds that are too hydrophobic and generate too much acidity to be used as a stabilizing drug matrix for insulin can be used as release retarding compositions to control the release of a hydrophilic, water soluble drug such as insulin.

A base or cap deposit can be a second drug to treat a second condition, so the deposit can fulfill two functions simultaneously. When one drug is to be released rapidly luminally and a second drug is to be released slowly murally, such as is the case with insulin and Pimecrolimus, Pimecrolimus proved to be an excellent cap to control the directional release of insulin, and it was also the drug of choice for slow mural release.

The hydrophobic non-polymer compositions which function in the present invention to retard or substantially prevent release of water soluble drugs are combined with 50% or less binder, preferably 30% or less, and often even 10% or less. The outer portion of the hydrophobic compound and binder forms a generally solid structure with a glass transition or melting point temperature of 37° C. or greater. The hydrophobic non-polymeric compound can also be a blend of two or more such compounds. Although a polymer binder is described herein, it should be understood that the binder can be omitted where the hydrophobic compound itself forms a sufficiently solid structure to be retained in the openings 14. The binder is a non-water soluble polymer which can be hydrophobic or hydrophilic.

If the hydrophobic non-polymeric compound is neutral, it will have an octanol/water partition value P such that Log P is equal to or greater than one. If the hydrophobic non-polymeric component is acidic or basic, or is ionic, either as an anion or cation, it will have an octanol/water distribution value D such that Log D at pH 7.4 is equal to or greater than one.

Both the inner portion 30 and the outer portion 40 are preferably amorphous, or at least predominantly amorphous with a minor amount of a crystalline second phase. Non-polymeric components that have crystalline melting points can be admixed with one or more non-polymeric or polymeric components such that the final formulated composition is amorphous, or at least predominantly amorphous with a minor amount of a crystalline second phase. Hydrophobic non-polymeric compounds that are liquid at ambient temperature can be mixed with crystalline non-polymeric components or polymeric components such that the final composition is amorphous and has a glass transition temperature of 37° C. or greater. Preferably, the liquid hydrophobic component has a boiling point above 150° C., more preferably above 200° C.

The hydrophobic non-polymeric compound may itself be a drug or other therapeutic agent, different from the water soluble drug, and having a Log P or Log D value of one or greater. Examples include pimecrolimus, sirolimus, everolimus, ABT-578, farglitizar, Imatinib, dexamethasone, probucol, rosigitazone, pioglitazoneand paclitaxel. Preferably, hydrophobic drug compounds will be admixed with 5% or more of a non-water soluble polymer to act as a binder.

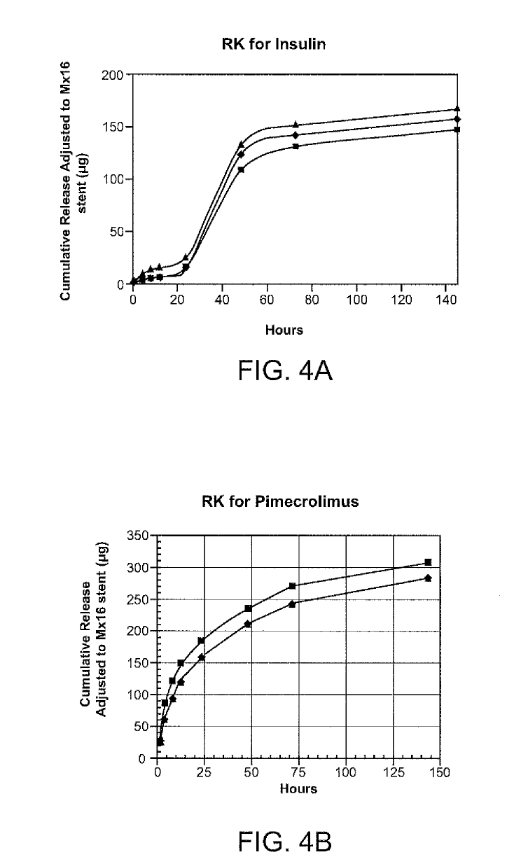

One example of the use of a drug as a hydrophobic non-polymeric outer portion to retard release is Pimecrolimus. Pimecrolimus can be combined with a minor proportion of PLGA polymer (5-30%) as a murally located deposits for an insulin inner portion in a stent. Examples of insulin and Pimecrolimus stents are described below in Examples 1-6 and shown in FIGS. 4-9

The hydrophobic non-polymeric compound can be various other non-drug materials, such as preservative, additives, antioxidants, plasticizers, and stabilizers.

Examples of solid hydrophobic non-polymeric compounds include butylated hydroxy toluene (BHT), butylated hydroxy anisole (BHA), methyl 4-hydroxybenzoate, propyl 4-hydroxybenzoate, butyl 4-hydroxybenzoate. All these components are themselves crystalline solids, so it is envisioned that they can be used with or without polymer to form an amorphous formulation.

Examples of liquid hydrophobic non-polymeric compounds include acetyl tributylcitrate (ATBC), benzyl benzoate, ethyl benzoate, benzyl alcohol. It is envisioned that these liquid compounds would be used with a polymer or other binder to form an amorphous formulation. The liquid and solid non-drug hydrophobic compounds can be mixed together or mixed with drugs in the outer portion 40.

Examples of non-water soluble bioresorbable polymers which can be used as binders for the hydrophobic compounds include polylactic acid (PLA) or polylactic-co-glycolic acid (PLGA), polycaprolactone (PCL), polylactic polycaprolactone (PLA-PCL) copolymers, poly(anhydride), poly(orthoester), poly(alpha-hydroxy acid) polymer (a “PHA” polymer, such as poly(hydroxybutyrate), poly(hydroxyvalerate, or poly(hydroxybutryate-co-hydroxyvalerate), a poly(beta-hydroxy acid), an aliphatic poly(carbonate) or ester-carbonate copolymer, such as PLA-TMC. Binders can also be non-bioresorbable polymers or non-polymers.

Examples of hydrophobic non-polymeric compounds are given in Table 1 with their calculated Log P or Log D octanol to water partition coefficients.

TABLE 1

| | Calculated | | | |
|---|---|---|---|---|
| | Log P | Liquid/ | Percent of Compound | |
| Compound | octanol/water | Solid | in Water Phase | |
| Probucol | 10.72 | S | 2.0E−09 | Most |
| | | | | Hydrophobic |
| Pimecrolimus | 6.99 | S | 1.02E−05 | |
| Sirolimus | 5.5 | S | 3.16E−04 | |
| Midostaurin | 5.5 | S | 3.3E−04 | |
| BHT | 5.03 | S | 0.001 | |
| Farglitizar (pH 5.8) | 4.53 | S | 0.003 | |
| ATBC | 4.29 | L | 0.005 | |
| Imatinib | 4.18 | S | 0.007 | |
| Paclitaxel | 3.62 | S | 0.024 | |
| Benzyl Benzoate | 3.54 | L | 0.029 | |
| BHA | 3.50 | S | 0.032 | |
| Butyl 4- | 3.47 | S | 0.034 | |
| Hydroxybenzoate | | | | |
| Tranilast | 3.27 | S | 0.054 | |
| Phenyl 4- | 3.21 | S | 0.062 | |
| Hydroxybenzoate | | | | |
| Propyl 4- | 2.98 | S | 0.10 | |
| hydroxybenzoate | | | | |
| Ethyl Benzoate | 2.32 | L | 0.48 | |
| Anisole | 2.07 | L | 0.84 | |
| Methyl 4- | 2.00 | S | 0.99 | |
| Hydroxybenzoate | | | | |
| Dexamethasone | 1.77 | S | 1.7 | |
| Farglitizar (ph 7.4) | 1.19 | S | 6.1 | |
| Benzyl alcohol | 1.08 | L | 7.7 | Least |
| | | | | Hydrophobic |

Examples of the proportions of non-polymeric solids, liquids and polymer that give amorphous mixtures are shown in Table 2.

TABLE 2

| | | Film Morphology | | | | | |
|---|---|---|---|---|---|---|---|
| Release | | Percent Agent in PLGA 85/15 Film Cast | | | | | |
| Suppression | Agent | from Anisole | | | | | |
| Agent | Form | 5% | 10% | 25% | 50% | 75% | 90% |
| BHT | Solid | A | A | A | D | Mixed | C |
| | | | | | | A/C | |
| BHT:BHA::50:50 | Solid | — | — | — | A | A | — |
| BHT:BHA::55:45 | Solid | — | — | — | — | — | A |
| Butyl 4- | Solid | A | A | A | C | C | C |
| Hydroxybenzoate | | | | | | | |
| (Butyl Paraben) | | | | | | | |
| Propyl 4- | Solid | A | A | Mixed | C | C | C |
| Hydroxybenzoate | | | | A/C | | | |
| (Propyl Paraben) | | | | | | | |
| Benzyl Benzoate | Liquid | A | A | — | — | — | — |
| Acetyl Tributyl | Liquid | A | A | — | — | — | — |
| Citrate (ATBC) | | | | | | | |
| Pimecrolimus | Solid | — | A | Mixed | C | — | — |
| | | | | A/C | | | |

A—Amorphous
D—Dispersion
C—Crystalline

Examples of water soluble drugs whose release rate from a stent reservoir will be retarded by employing the method and composition of the invention include insulin, Angiomax, dipyridamole, Gleevec (imatinib mesylate), cladribine (2-CdA), heparin, aspirin, doxycycline and doxycycline hyclate. Generally, water soluble drugs for the purpose of release from an implantable medical device are drugs whose solubility in water is greater than about 0.1 mg per milliliter. Even drugs with low water solubilities such as cladribine (0.2 mg/ml) are difficult to hold back when placed within the high water environment of the body.

### EXAMPLE 1

A stent is loaded with the insulin arranged for luminal delivery and Pimecrolimus arranged for mural delivery and tested in the following procedure. A first mixture of poly(lactide-co-glycolide) (PLGA) and a suitable organic solvent, such as DMSO, NMP, or anisole is prepared. The mixture is loaded dropwise into holes in the stent then the solvent is evaporated to begin formation of a base region without drug. The loading of PLGA is repeated to form a desired base.

A second mixture of PEVA and a suitable organic solvent are then introduced into the holes and the solvent is evaporated to complete the base region.

A third mixture of insulin and PLGA, in a suitable organic solvent such as DMSO or NMP is introduced into holes in the stent over the base. The solvent is evaporated to form an insulin deposit and the filling and evaporation procedure is repeated until the total dosage of insulin is about 250 micrograms for a 3 mm×16 mm stent. Equivalent dosages are used on stents of other sizes.

A fourth solution, of PEVA and a suitable organic solvent, such as DMSO, is then laid down over the insulin deposit.

A fifth solution of Pimecrolimus and PLGA in a suitable organic solvent is then laid down and repeated until the total dosage of Pimecrolimus is about 300 micrograms.

A final solution of PLGA mixed with PLA-PCL copolymer in a suitable organic solvent is then laid down to complete the cap or outer portion.

The resulting stent is tested in an in vitro test system which is described below in Example 7 and the release for insulin and Pimecrolimus are shown in FIG. 4. As shown in FIG. 4A, the insulin release follows an S-shape release curve with a slow initial release increasing after about 20 hours and then slowing after about 40 hours. As shown in FIG. 4B, the Pimecrolimus release includes a release of greater than 50% at about 24 hours slowing after 24 hours.

### EXAMPLE 2

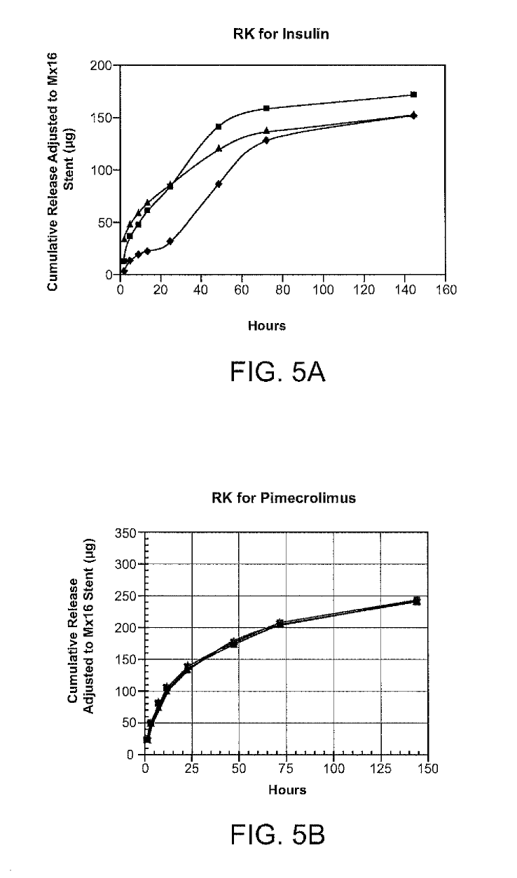

Another stent is loaded with insulin and Pimecrolimus as in Example 1, except that an additional deposit of PLGA/PLA-PCL copolymer is added between the fourth and fifth solutions. The resulting stent is tested in the in vitro test system and the release for insulin and Pimecrolimus are shown in FIG. 5.

### EXAMPLE 3

Another stent is loaded with insulin and Pimecrolimus as in Example 1, except that the base deposit includes part PLGA and another part PCL and the cap deposits include a first deposit of PCL and two different drug to polymer ratios of Pimecrolimus in PLGA. A first portion of the Pimecrolimus deposit has a ratio of drug to polymer of about 75:25 while a second portion of the Pimecrolimus deposit has a ratio of drug to polymer of about 95:5. The higher concentration of the Pimecrolimus closer to the luminal end of the stent reservoirs allows the initial release of drug in the first 24 hours to be increased.

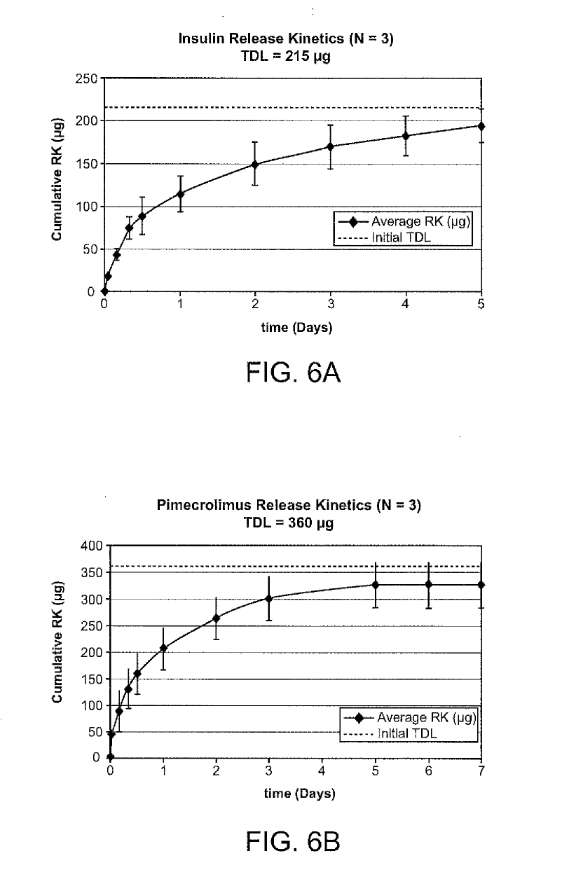

The total drug load was 215 micrograms of insulin and 360 micrograms of Pimecrolimus. The resulting stent is tested in the in vitro test system and the release for insulin and Pimecrolimus are shown in FIG. 6.

### EXAMPLE 4

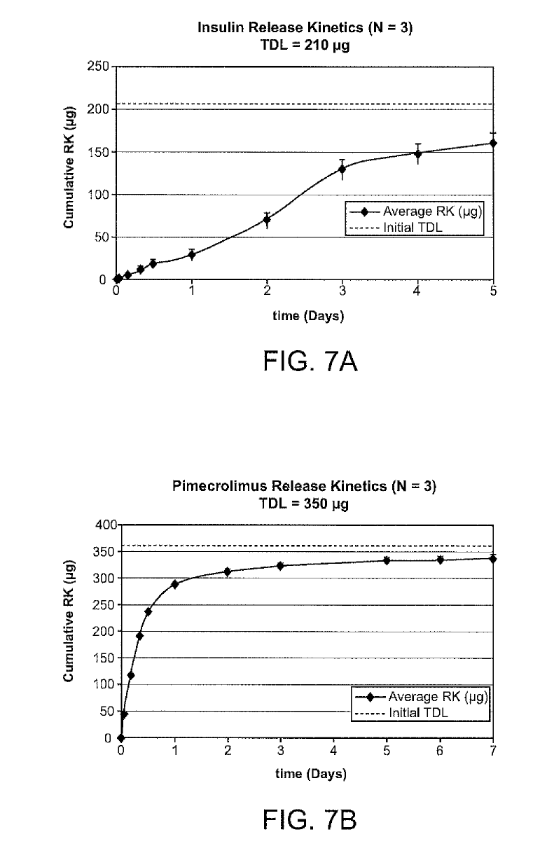

Another stent is loaded with insulin and Pimecrolimus as in Example 3, except that the PCL in the base and cap deposits is replaced with PEVA. The resulting stent is tested in the in vitro test system and the release for insulin and Pimecrolimus are shown in FIG. 7.

### EXAMPLE 5

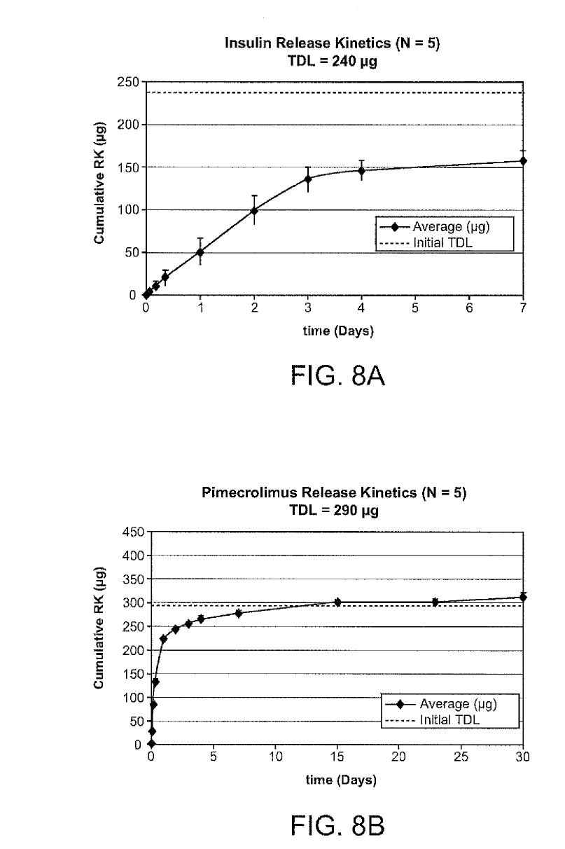

Another stent is loaded with insulin and Pimecrolimus as in Example 5, except that the PEVA in the cap deposit is replaced with a mixture of PLGA/PLA-PCL copolymer. The resulting stent is tested in the in vitro test system and the release for insulin and Pimecrolimus are shown in FIG. 8.

### EXAMPLE 6

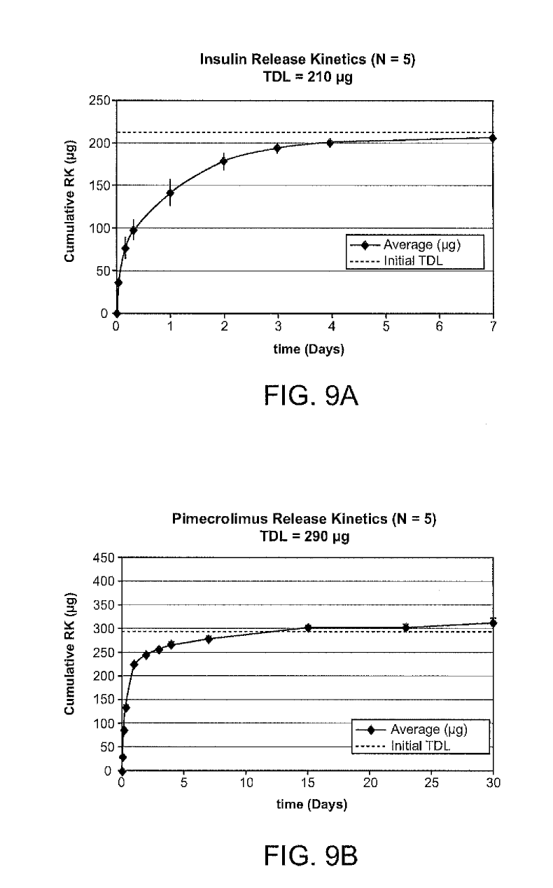

Another stent is loaded with insulin and Pimecrolimus as in Example 3, except that the PLGA/PLA-PCL copolymer in the base and cap deposits is replaced with PLGA. The resulting stent is tested in the in vitro test system and the release for insulin and Pimecrolimus are shown in FIG. 9. FIG. 9A shows a release of between 60-80% of the insulin in the first day and a release of 70-90% of the Pimecrolimus in the first day followed by a slow extended release over at least 30 days.

### EXAMPLE 7

The following is the in vitro test procedure for generating the release curves for insulin and Pimecrolimus in the Examples. The elution rates of drug from the Examples above are determined in a standard sink condition experiment.

The total drug load (TDL) of insulin from a stent is determined by extracting all the polymer and drug from the stent in the solvent dimethyl sulfoxide (DMSO). The amount of insulin in a solution sample is determined by High Pressure Liquid Chromatography (HPLC). The following conditions are used:

Analysis Column: Discovery BIO Wide Pore C5 HPLC Column (150 mm×4.6 mm 5 micron particle)

Mobile phase. Water/Acetonitrile::68% vol./32% vol.

Flow Rate: 1.0 mL/minute

Temperature: 25° C. ambient

Detection wavelength: 214 nm

Injection volume: 20 μL

Retention time: 7 minutes

The in vitro release kinetic (RK) for insulin from a stent is determined by placing the stent in a vial with a release solution for a period of time, removing the stent and placing the stent into fresh vial of the release solution for a period of time, and repeating this procedure for all time points.

The release solution for measurement of RK is a solution of phosphate buffered saline (PBS) prepared by dissolving five “Phosphate Buffered Saline Tablets” (Sigma-Aldrich Co.) in 1000 mL deionized water to provide a solution with a pH of 7.4, 0.01 M in phosphate buffer, 0.0027 M in potassium chloride and 0.137 M in sodium chloride.

The amount of insulin in the RK samples is determined by High Pressure Liquid Chromatography (HPLC) with the conditions described above. By comparison with a calibration curve generated from known stock solutions, the amount of insulin eluted into the release solution during any time period of the experiment can be calculated.

The total drug load (TDL) of Pimecrolimus from a stent is determined by extracting all the polymer and drug from the stent in the solvent acetonitrile. The amount of Pimecrolimus in a solution sample is determined by HPLC. The following conditions are used:

Analysis Column: Chromolith (100 mm×4.6 mm 3 micron RP-E)

Mobile phase: Water/Acetonitrile::68% vol./32% vol.

Flow Rate: 1.5 mL/minute

Temperature: 50° C.

Detection wavelength: 194 nm

Injection volume: 30 μL

Retention time: 15 minutes

The in vitro release kinetic (RK) for Pimecrolimus from a stent is determined by placing the stent in a vial with a release solution for a period of time, removing the stent and placing the stent into fresh vial of the release solution for a period of time, and repeating this procedure for all time points.

The release solution for measurement of RK is a solution of propylene glycol 40% and pH5 acetate buffer 60%. The amount of Pimecrolimus in the RK samples is determined by HPLC with the conditions described above. By comparison with a calibration curve generated from known stock solutions, the amount of Pimecrolimus eluted into the release solution during any time period of the experiment can be calculated.

While the invention has been described in detail with reference to the preferred embodiments thereof it will be apparent to one skilled in the art that various changes and modifications can be made and equivalents employed, without departing from the present invention.

What is claimed is:

1. An implantable drug delivery system for retarding release of water soluble drugs, the system comprising:
an inner portion of the drug delivery system comprising a water soluble drug and a drug matrix material which stabilizes the drug; and
an outer portion of the drug delivery system which retards the release of the water soluble drug from the inner portion, the outer portion comprising a hydrophobic non-polymer compound and less than 50% of a binder, wherein when the drug delivery system is implanted in a body the outer portion retards the release of the water soluble drug by controlling fluid passing from the body into the inner portion and by controlling passage of the water soluble drug from the inner portion into the body.

2. The system of claim 1, wherein the water soluble drug has a solubility in water of greater than 0.1 mg/ml.

3. The system of claim 2, wherein the water soluble drug is insulin.

4. The system of claim 2, wherein the water soluble drug is an antirestenotic.

5. The system of claim 2, wherein the water soluble drug is selected from the group of Angiomax, dipyridamole, imatinib mesylate, cladribine, heparin, aspirin, doxycycline, and doxycycline hyclate.

6. The system of claim 1, wherein the drug matrix material is hydrophilic.

7. The system of claim 1, wherein the drug matrix material is biodegradable.

8. The system of claim 7, wherein the drug matrix material is a polymer.

9. The system of claim 1, wherein the drug matrix material is a polymer.

10. The system of claim 9, wherein the drug matrix material is polylactic acid or a copolymer thereof.

11. The system of claim 10, wherein the drug matrix material is polylactic-co-glycolic acid.

12. The system of claim 1, wherein hydrophobic material is a drug.

13. The system of claim 12, wherein the drug is an antirestenotic drug.

14. The system of claim 13, wherein the drug is pimecrolimus, sirolimus, everolimus, ABT-578, or paclitaxel.

15. The system of claim 1, wherein the hydrophobic material has a calculated Log P or Log D value of at least one.

16. The system of claim 15, wherein the hydrophobic material is a drug.

17. The system of claim 15, wherein the hydrophobic material is a preservative or plasticizer.

18. The system of claim 1, wherein the hydrophobic material has a molecular weight of less than 3000.

19. The system of claim 18, wherein the hydrophobic material is a drug.

20. The system of claim 18, wherein the hydrophobic material is a preservative or a plasticizer.

21. The system of claim 1, wherein the outer portion comprises the hydrophobic material and less than 30% of the binder.

22. The system of claim 1, wherein the outer portion comprises the hydrophobic material and less than 10% of the binder.

23. The system of claim 12, wherein the outer portion comprises the hydrophobic material and less than 30% of the binder.

24. The system of claim 12, wherein the outer portion comprises the hydrophobic material and less than 10% of the binder.

25. A drug delivery stent comprising:
an expandable stent structure having a plurality of reservoirs;
a drug delivery system provided within the reservoirs of the stent structure, the drug delivery system having an inner portion and an outer portion;
wherein the inner portion of the drug delivery system comprises a water soluble drug and a drug matrix material which stabilizes the drug; and
wherein the outer portion of the drug delivery system retards the release of the water soluble drug from the inner portion, the outer portion comprising a hydrophobic non-polymer compound and of a binder at a ratio of less than 50% by weight of the binder, wherein when the stent is implanted in a body the outer portion retards the release of the water soluble drug by controlling fluid passing from the body into the inner portion and by controlling passage of the water soluble drug from the inner portion into the body.

26. The stent of claim 25, wherein the water soluble drug has a solubility in water of greater than 1 mg/ml.

27. The stent of claim 26, wherein the water soluble drug is insulin.

28. The stent of claim 26, wherein the water soluble drug is an antirestenotic.

29. The stent of claim 26, wherein the water soluble drug is selected from the group of Angiomax, dipyridamole, imatinib mesylate, cladribine, heparin, aspirin, doxycycline, and doxycycline hyclate.

30. The stent of claim 25, wherein the drug matrix material is hydrophilic.

31. The stent of claim 25, wherein the drug matrix material is biodegradable.

32. The stent of claim 31, wherein the drug matrix material is a polymer.

33. The stent of claim 25, wherein the drug matrix material is a polymer.

34. The stent of claim 33, wherein the drug matrix material is polylactic acid or a copolymer thereof.

35. The stent of claim 34, wherein the drug matrix material is polylactic-co-glycolic acid.

36. The stent of claim 25, wherein hydrophobic material is a drug.

37. The stent of claim 36, wherein the drug is an antirestenotic drug.

38. The stent of claim 37, wherein the drug is pimecrolimus, sirolimus, everolimus, ABT-578, or paclitaxel.

39. The stent of claim 25, wherein the hydrophobic material has a calculated Log P or Log D value of at least one.

40. The stent of claim 39, wherein the hydrophobic material is a drug.

41. The system of claim 39, wherein the hydrophobic material is a preservative or plasticizer.

42. The stent of claim 25, wherein the hydrophobic material has a molecular weight of less than 3000.

43. The stent of claim 42, wherein the hydrophobic material is a drug.

44. The stent of claim 42, wherein the hydrophobic material is a preservative or a plasticizer.

45. The stent of claim 25, wherein the outer portion forms a cap over the inner portion within the reservoir.

46. The stent of claim 45, wherein the inner portion and the outer portion are both formed entirely within the reservoirs.

47. The stent of claim 25, wherein the water soluble drug is insulin and the hydrophobic compound is an antirestenotic.

48. A drug delivery stent comprising:
an expandable stent structure having a plurality of reservoirs; a drug delivery system provided within the reservoirs of the stent structure, the drug delivery system having an inner portion and an outer portion;
wherein the inner portion of the drug delivery system comprises a water soluble drug and a drug matrix material which stabilizes the drug; and
wherein the outer portion of the drug delivery system retards the release of the water soluble drug from the inner portion, the outer portion comprising a hydrophobic non-polymer compound and of a binder at a ratio of less than 50% by weight of the binder, wherein when the stent is implanted in a body the outer portion retards the release of the water soluble drug by controlling fluid passing from the body into the inner portion and by controlling passage of the water soluble drug from the inner portion into the body.

49. The stent of claim 48, wherein hydrophobic material is a drug.

50. The stent of claim 48, wherein the outer portion comprises the hydrophobic material and less than 30% of the binder.

51. The stent of claim 48, wherein the outer portion comprises the hydrophobic material and less than 10% of the binder.

52. A drug delivery stent comprising:
an expandable stent structure;
a drug delivery system secured to the stent structure, the drug delivery system having an inner portion and an outer portion; wherein the inner portion of the drug delivery system comprises a water soluble drug and a drug matrix material which stabilizes the drug; and
wherein the outer portion of the drug delivery system retards the release of the water soluble drug from the inner portion, the outer portion comprising a hydrophobic non-polymer compound, wherein when the stent is implanted in a body the outer portion retards the release of the water soluble drug by controlling fluid passing from the body into the inner portion and by controlling passage of the water soluble drug from the inner portion into the body.

53. The stent of claim 52, wherein the water soluble drug is insulin.

54. The stent of claim 53, wherein hydrophobic material is a drug.

55. The stent of claim 54, wherein the drug is an antirestenotic drug.

56. The stent of claim 55, wherein the drug is pimecrolimus, sirolimus, everolimus, ABT-578, or paclitaxel.

57. The stent of claim 56, wherein the outer portion comprises the hydrophobic material and less than 30% of the binder.

58. The stent of claim 56, wherein the outer portion comprises the hydrophobic material and less than 10% of the binder.
